(19) United States
(12) Patent Application Publication
(10) Pub. No.: US 2022/0208330 A1
(43) Pub. Date: Jun. 30, 2022

(54) VISUALIZING OUTCOMES OF APPLYING RECOMMENDED INSULIN THERAPY SETTINGS, AND RELATED SYSTEMS AND DEVICES

(71) Applicant: Bigfoot Biomedical, Inc., Milpitas, CA (US)

(72) Inventors: Bethany Leigh Salmon, San Jose, CA (US); KC Choi, Milpitas, CA (US); Bryan Mazlish, Palo Alto, CA (US); Berkley Nelson, Milpitas, CA (US)

(21) Appl. No.: 17/646,568

(22) Filed: Dec. 30, 2021

Related U.S. Application Data
(60) Provisional application No. 63/132,871, filed on Dec. 31, 2020.

(51) Int. Cl. G16H 20/17

(52) U.S. Cl. CPC G16H 20/17

(57) ABSTRACT

Visualization of change of quality of glucose control is discussed, as well as systems, methods and devices for performing the same. In one or more examples, graphical representations of changes in quality of glucose control are combined with representations of changes to therapy settings for visualization of outcomes of applying therapy-setting updates to an insulin therapy of a patient. Such therapy setting updates may be provided by an insulin therapy management system, a health care provider, a caregiver, or a patient.

### CROSS-REFERENCE TO RELATED APPLICATION

This application claims the benefit under 35 U.S.C. § 119(e) of U.S. Provisional Patent Application Ser. No. 63/132,871, filed Dec. 31, 2020, the disclosure of which is hereby incorporated herein in its entirety by this reference.

### FIELD

Examples discussed herein relate, generally, to insulin therapy and visualizing outcomes of applying recommended insulin therapy settings. More specifically, one or more examples relate to visualizing outcomes of applying insulin therapy settings recommended by an insulin therapy management system, a health care provider, or a patient.

### BACKGROUND

Diabetes mellitus is a chronic metabolic disorder caused by the inability of a person's pancreas to produce sufficient amounts of the hormone insulin such that the person's metabolism is unable to provide for the proper absorption of sugar and starch. The inability to absorb those carbohydrates sometimes leads to hyperglycemia, i.e., the presence of an excessive amount of glucose within the blood plasma. Hyperglycemia has been associated with a variety of serious symptoms and life threatening long-term complications such as dehydration, ketoacidosis, diabetic coma, cardiovascular diseases, chronic renal failure, retinal damage and nerve damages with the risk of amputation of extremities.

Because healing is not yet possible, a permanent therapy is necessary which maintains a proper blood glucose level within normal limits. Maintaining a proper glucose level is achieved by regularly supplying insulin to a person with diabetes (PWD). Excessive insulin delivery can result in acute hypoglycemia, which has been associated with a variety of serious symptoms, long-term complications, and death.

Historically, diabetes is treated with multiple, daily injections of rapid and long acting insulin via a hypodermic syringe. One or two injections per day of a long acting insulin is administered to provide a basal level of insulin and additional injections of a rapidly acting insulin is administered before or with each meal in an amount proportional to the size of the meal. Insulin therapy can also be administered using an insulin pump that provides periodic or continuous release of the rapidly acting insulin to provide for a basal level of insulin and larger doses of that same insulin at the time of meals. Insulin pumps allow for the delivery of insulin in a manner that bears greater similarity to the naturally occurring physiological processes and can be controlled to follow standard or individually modified protocols to give the patient better glycemic control. In some circumstances, an insulin pump device can store (via input from a clinician or a user) a number of settings (e.g., dosage parameters or other settings) that are customized by the physician for the particular user.

PWDs, their caregivers, and their health care providers (HCPs) bear a great deal of cognitive burden in managing intensive medicine therapy. Delivering the correct amount of the medicine at the correct time is an extremely challenging endeavor. Such delivery requires the patient to make dosing determinations multiple times per day and also requires a combination of the patient and the HCP to recalibrate the therapeutic parameters of the therapy on an episodic time frame that varies from individual to individual, and within individuals based on age and/or behavior (e.g., change in exercise, change in diet).

An HCP (e.g., physician, endocrinologist, without limitation) may assist a PWD in the self-treatment. For example, an HCP may assist a PWD by providing therapy recommendations personally or via a dosing system used to connect PWDs with HCPs to improve awareness and knowledge with the goal to ultimately improve insulin therapy outcomes. Some conventional dosing systems provide recommendations to PWDs and HCPs for updating and changing insulin therapy settings and/or track blood glucose patterns, carbohydrate intake, and exercise, and provide summary information of the same. Some conventional dosing systems focus on type 2 diabetes with an emphasis on only basal rate, requesting food logging, requesting exercise logging, and information about past blood glucose highs and lows.

Although conventional dosing systems may remove some of the mental burdens for the HCP and/or PWD in determining an appropriate recommendation related to insulin dosing, dosing systems still burden the HCP and/or PWD with the mental task of at least manually evaluating therapy data, manually determining a dosing recommendation, manual entry of data, and evaluating the outcome tradeoffs associated with adopting a therapy setting recommendation.

### BRIEF DESCRIPTION OF THE DRAWINGS

To easily identify the discussion of any particular element or act, the most significant digit or digits in a reference number refer to the figure number in which that element is first introduced.

FIG. 1 is a block diagram depicting an insulin therapy management system in accordance with one or more examples.

FIG. 2A illustrates a display portion in accordance with one or more examples.

FIG. 2B illustrates a display portion in accordance with one or more examples.

FIG. 2C illustrates a display portion in accordance with one or more examples.

FIG. 3 illustrates a computing system in accordance with one or more examples.

FIG. 4 illustrates a process for generating a graphical representation of changes in quality of glucose control in accordance with one or more examples.

FIG. 5 illustrates a computing apparatus in accordance with one or more examples.

FIGS. 6A and 6B illustrate a process for visualizing changes in quality of glucose control in accordance with one or more examples.

### DETAILED DESCRIPTION

In the following detailed description, reference is made to the accompanying drawings, which form a part hereof, and in which are shown, by way of illustration, specific example examples in which the present disclosure may be practiced. These examples are described in sufficient detail to enable a person of ordinary skill in the art to practice the present disclosure. However, other examples may be utilized, and structural, material, and process changes may be made without departing from the scope of the disclosure.

The following description may include examples to help enable one of ordinary skill in the art to practice the disclosed examples. The use of the terms “exemplary,” “by example,” and “for example,” means that the related description is explanatory, and though the scope of the disclosure is intended to encompass the examples and legal equivalents, the use of such terms is not intended to limit the scope of an example, or this disclosure, to the specified components, steps, features, functions, or the like.

It will be readily understood that the components of the examples as generally described herein and illustrated in the drawings could be arranged and designed in a wide variety of different configurations. Thus, the following description of various examples is not intended to limit the scope of the present disclosure, but is merely representative of various examples. While the various aspects of the examples may be presented in the drawings, the drawings are not necessarily drawn to scale unless specifically indicated.

Furthermore, specific implementations shown and described are only examples and should not be construed as the only way to implement the present disclosure unless specified otherwise herein. Elements, circuits, and functions may be shown in block diagram form in order not to obscure the present disclosure in unnecessary detail. Conversely, specific implementations shown and described are exemplary only and should not be construed as the only way to implement the present disclosure unless specified otherwise herein. Additionally, block definitions and partitioning of logic between various blocks is exemplary of a specific implementation. It will be readily apparent to one of ordinary skill in the art that the present disclosure may be practiced by numerous other partitioning solutions. For the most part, details concerning timing considerations and the like have been omitted where such details are not necessary to obtain a complete understanding of the present disclosure and are within the abilities of persons of ordinary skill in the relevant art.

Those of ordinary skill in the art will understand that information and signals may be represented using any of a variety of different technologies and techniques. Some drawings may illustrate signals as a single signal for clarity of presentation and description. It will be understood by a person of ordinary skill in the art that the signal may represent a bus of signals, wherein the bus may have a variety of bit widths and the present disclosure may be implemented on any number of data signals including a single data signal.

The various illustrative logical blocks, modules, and circuits described in connection with the examples disclosed herein may be implemented or performed with a general purpose processor, a special purpose processor, a digital signal processor (DSP), an Integrated Circuit (IC), an Application Specific Integrated Circuit (ASIC), a Field Programmable Gate Array (FPGA) or other programmable logic device, discrete gate or transistor logic, discrete hardware components, or any combination thereof designed to perform the functions described herein. A general-purpose processor (may also be referred to herein as a host processor or simply a host) may be a microprocessor, but in the alternative, the processor may be any conventional processor, controller, microcontroller, or state machine. A processor may also be implemented as a combination of computing devices, such as a combination of a DSP and a microprocessor, a plurality of microprocessors, one or more microprocessors in conjunction with a DSP core, or any other such configuration. A general-purpose computer including a processor is considered a special-purpose computer while the general-purpose computer is configured to execute computing instructions (e.g., software code) related to examples of the present disclosure.

The examples may be described in terms of a process that is depicted as a flowchart, a flow diagram, a structure diagram, or a block diagram. Although a flowchart may describe operational acts as a sequential process, many of these acts can be performed in another sequence, in parallel, or substantially concurrently. In addition, the order of the acts may be re-arranged. A process may correspond to a method, a thread, a function, a procedure, a subroutine, a subprogram, other structure, or combinations thereof. Furthermore, the methods disclosed herein may be implemented in hardware, software, or both. If implemented in software, the functions may be stored or transmitted as one or more instructions or code on computer-readable media. Computer-readable media includes both computer storage media and communication media including any medium that facilitates transfer of a computer program from one place to another.

It should be understood that any reference to an element herein using a designation such as “first,” “second,” and so forth does not limit the quantity or order of those elements, unless such limitation is explicitly stated. Rather, these designations are used herein as a convenient method of distinguishing between two or more elements or instances of an element. Thus, a reference to first and second elements does not mean that only two elements can be employed or that the first element must precede the second element in some manner. Also, unless stated otherwise a set of elements may comprise one or more elements. Likewise, sometimes elements referred to in the singular form may also include one or more instances of the element.

A period of time for which a graphical representation depicts changes in quality of a patient's glucose control is referred to herein as a “period-of-interest.”

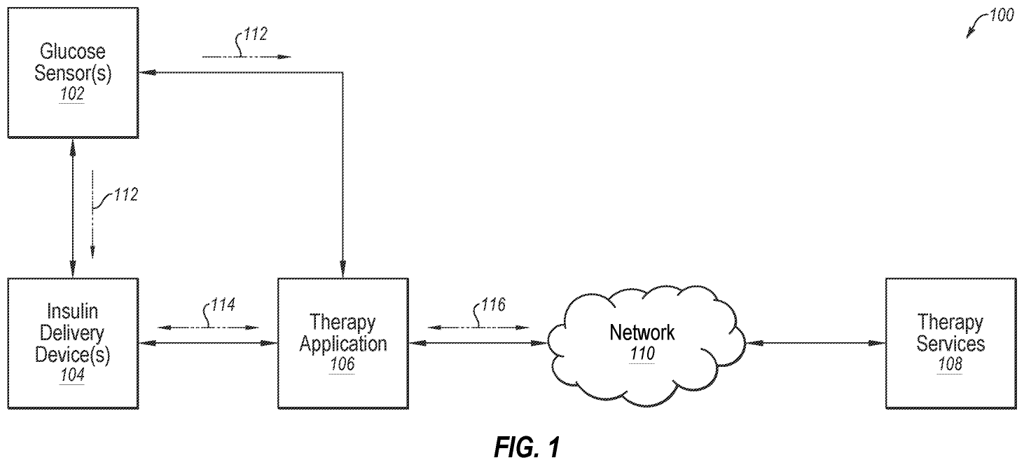

FIG. 1 is a block diagram depicting an insulin therapy management system 100 for managing diabetes, in accordance with one or more examples. Insulin therapy management system 100 may include glucose sensor(s) 102, insulin delivery device(s) 104, therapy application 106 and therapy services 108.

Glucose sensor(s) 102 is configured to generate sensor data 112 associated with blood glucose measurements taken from the body of a patient. In various examples, glucose sensor(s) 102 may be a continuous glucose monitor (CGM), a flash glucose monitor, a blood glucose meter (BGM), or any other suitable sensor. In the case of CGMs and flash glucose monitors, they may be configured to provide glucose data based on interstitial fluid glucose levels of a user, which may be correlated to blood glucose levels. In the case of a BGM, it may be configured to provide blood glucose data, typically based on a blood sample. Accordingly, while the term “blood glucose” may, at times, be used as a general term simply for convenience, the disclosure is not limited to using just blood glucose data, values, levels, etc., but also interstitial fluid glucose levels, as well as any intermediate measurement values. The terms “blood glucose data” and “glucose data” are used interchangeably herein to refer to blood glucose data.

Insulin delivery device(s) 104 may be any manual, automated, semi-automated, or combinations thereof, device or system suitable for delivery of insulin to the body to manage diabetes. Non-limiting examples of manual insulin delivery devices include syringes, insulin pens, and insulin inhalers that can deliver rapid-acting or long-acting insulin. Non-limiting examples of insulin delivery devices suitable for automated and semi-automated insulin delivery include infusion pumps that deliver doses of rapid-acting insulin to the body in response to control signals. Such control signals (e.g., generated responsive to, or including, optional control settings 114 of FIG. 1, without limitation) may be generated by a controller according to a dosing schedule and/or in response to blood glucose data, meal information, lifestyle information, and physiological information. A controller may generate the control signals in an automated fashion (e.g., implementing control decisions based on an algorithm without requesting approval by a user (e.g., a patient, care giver, or healthcare provider), without limitation), semi-automated fashion (e.g., implementing control decisions based on an algorithm without requesting approval by a user under certain conditions and otherwise requesting approval before changing therapy settings or parameters, without limitation), or a supervised fashion (e.g., implementing a delivery schedule and delivering bolus doses or changing therapy settings (e.g., as determined by an algorithm, without limitation) only when approved by a user (e.g., a patient, health care provider, or care giver), without limitation).

Therapy application 106 may be a software application executing on a user device (e.g., mobile phone, tablet computer, wearable computer (e.g., a smart watch, without limitation), or personal computer, without limitation). Therapy application 106 may provide an interface for a user to view and/or interact with therapy data 116 about insulin therapy management, including without limitation, blood glucose data, meal data, insulin dosing data, therapy settings, therapy parameters, and therapy recommendations. In one or more examples, therapy application 106 may be in communication with therapy services 108 via network 110. Network 110 may be any combination of wired or unwired (e.g., wireless, without limitation) networks that permit electronic communication between therapy services 108 and therapy application 106. In one or more examples, user device upon which therapy application 106 executes may include communication equipment suitable for electronic communication with therapy services 108 via network 110. As a non-limiting example, therapy services 108 may be provided by applications executed on the cloud or a decentralized computing platform (e.g., a decentralized application, without limitation), accessed via the Internet, blockchain technology, or combinations thereof.

In one or more examples, therapy application 106 may be an interface between an insulin delivery system comprising glucose sensor(s) 102, insulin delivery device(s) 104 and therapy services 108. Insulin therapy management information, including therapy data 116, may be generated by therapy application 106 or may be generated by therapy services 108 and provided to therapy application 106 via network 110.

While features and functionalities are discussed with respect a glucose sensor(s) 102 and an insulin delivery device(s) 104, it is specifically contemplated that some or a totality of the features and functions are performed by accessory devices that connect to and/or monitor sensors and/or delivery devices, such as a pen cap, inhaler cap, or secure element (e.g., such an element may be, as a non-limiting example, an electronic device) that attaches to an insulin delivery device, without limitation.

One or more examples relate, generally, to depicting a graphical representation of change-in-quality of glucose control over time, for example, by an application such as therapy application 106 of FIG. 1, and related software applications and computing devices to execute the same. Various examples of graphical representations discussed herein enable visualization of change in quality of glucose control following insulin-setting changes, and such visualization may in turn may facilitate a user's appreciation for effect of insulin setting changes on quality of glucose control, anticipate effects of insulin setting changes on quality of glucose control, or both.

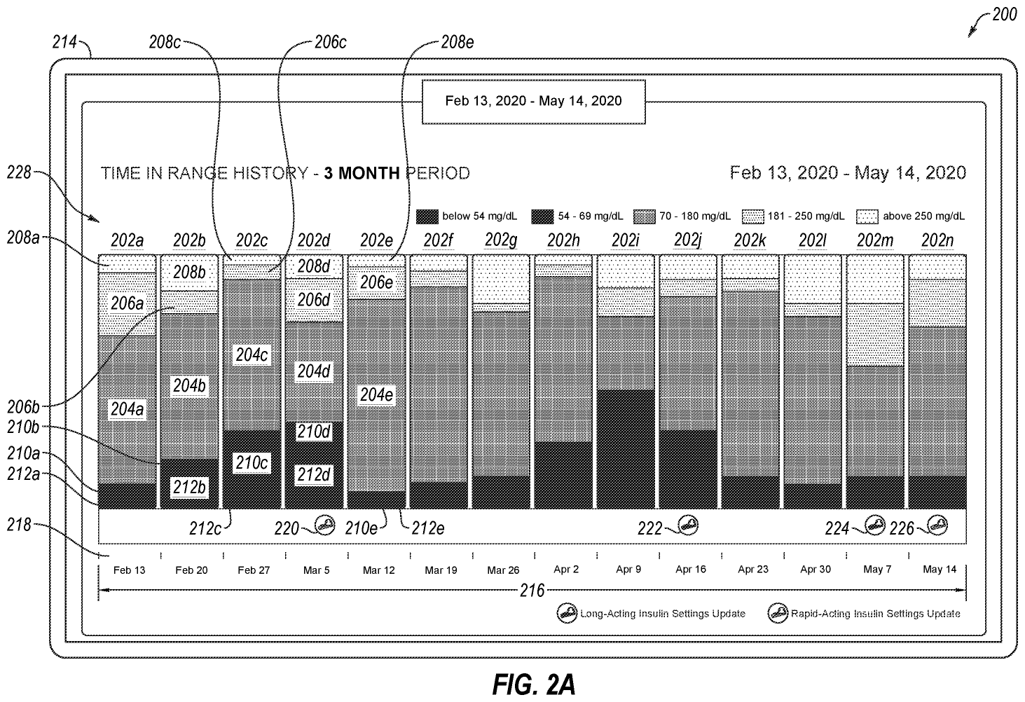

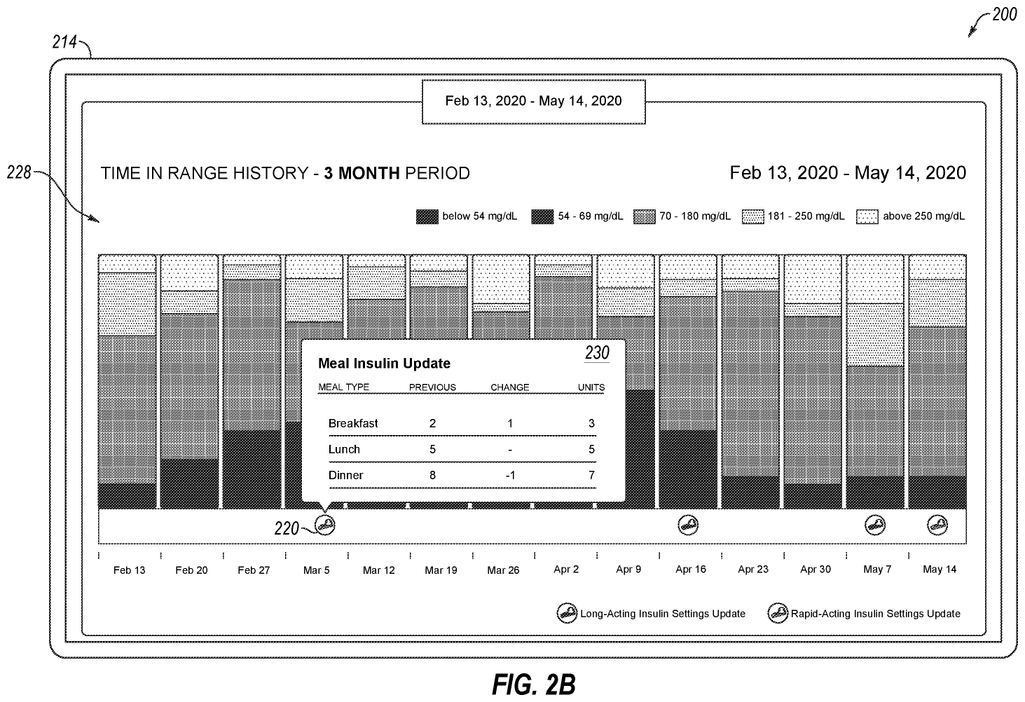

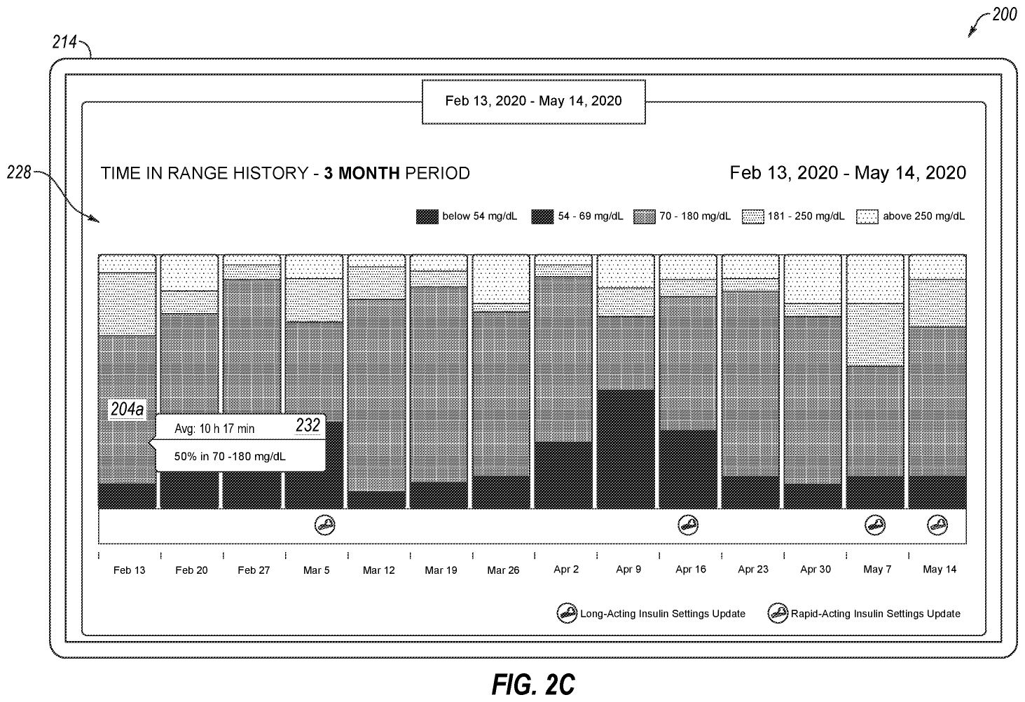

FIG. 2A is a diagram depicting a display portion 200 having a graphical user interface 214 overlaying a graphical representation 228 of changes in quality of a patient's glucose control over a period of time, such as a period of time referred to herein as a “period-of-interest.” By glancing from left to right (i.e., from the beginning of period-of-interest 216 to the end of period-of-interest 216, without limitation), a user may gain an appreciation of change in quality of glucose control over time for the specific patient associated with graphical user interface 214.

FIG. 2A depicts multiple stacked time-in-range (“TIR”) bar charts 202a, 202b, 202c, 202d, 202e, 202f, 202g, 202h, 202i, 202j, 202k, 202l, 202m, and 202n. For each stacked TIR, FIG. 2A depicts time-in bar charts, including: a level 2 hyper bar chart (e.g., level 2 hyper bar chart 208a-e, without limitation), level 1 hyper bar chart (e.g., level 1 hyper bar chart 206a-e, without limitation), in-range bar chart (e.g., in-range bar chart 204a-e, without limitation), level 1 hypo bar chart (e.g., level 1 hypo bar chart 210a-e, without limitation), and a level 2 hypo bar chart (e.g., level 2 hypo bar chart 212a-e, without limitation). FIG. 2A depicts time-in charts for only a subset of stacked TIRs 202a-n solely to avoid unnecessarily obscuring the depiction with additional labels.

A change in quality of glucose control is presented in graphical representation 228 by multiple stacked TIR bar charts (each instance of such a stacked TIR bar chart also referred to herein as a “TIR”) for period-of-interest 216. Each TIR represents quality of glucose control via a number of constituent bar charts that individually represent a specific quality factor of glucose control. Generally, a specific quality factor is a range of blood glucose levels associated with desirable or undesirable conditions. In the specific example depicted by FIG. 2A, ranges and associated conditions of quality factors are defined by Table 1:

| Blood glucose range | | Associated Condition |
|---|---|---|
| below 54 | mg/dL | Level 2 hypoglycaemia |
| 54-69 | mg/dL | Level 1 hypoglycaemia |
| 70-180 | mg/dL | Desired target range |
| 181-250 | mg/dL | Level 1 hyperglycaemia |
| above 250 | mg/dL | Level 1 hyperglycaemia |

Each TIR provides a visual representation (e.g., to a user, without limitation) of the relative contribution of a quality factor to an overall quality of glucose control represented by a given TIR. Portions of each TIR include bar charts, and constituent bar charts are individually associated with a quality factor. For example, TIR 202a includes in-range bar chart 204a, level 1 hyper bar chart 206a, level 2 hyper bar chart 208a, level 1 hypo bar chart 210a and level 2 hypo bar chart 212a. Positions of upper and lower thresholds of each portion may be adjusted in either vertical direction (e.g., up (e.g., toward the top of the respective stacked TIR when graphical user interface 214 is oriented the same as depicted by FIG. 2A, without limitation) or down (e.g., toward the bottom of the respective stacked TIR when graphical user interface 214 is oriented the same as depicted by FIG. 2A, without limitation), without limitation) such that the overall area of a bar chart is proportional to the relative contribution of an associated quality factor to the overall quality of glucose control represented by a TIR. FIG. 2A depicts level 1 hypo bar charts for TIR 202b, TIR, 202c, TIR 202d, and 202e, labelled as level 1 hypo bar chart 210b, 210c, 210d, and 210e, respectively.

For example, in TIR 202a, the areas of level 1 hypo bar chart 210a, level 2 hypo bar chart 212a and level 2 hyper bar chart 208a as compared to the areas of level 1 hyper bar chart 206a and in-range bar chart 204a reflect a smaller contribution to the quality of glucose control represented by TIR 202a. Notably, in-range bar chart 204a is presented at a location of TIR 202a between the other bar charts, and a user may visually compare in-range bar chart 204a to the other bar charts. Moreover, the location of in-range bar chart 204a facilitates appreciation by a user of the relative contribution of the “in-range” blood glucose levels to the overall quality of glucose control represented by TIR 202a.

In order to enable a user to gain an appreciation of the change in quality of glucose control for a patient over period-of-interest 216, a number of TIRs are presented, the presentation arranged sequentially (by time) over period-of-interest 216. Each TIR of graphical representation 228, in FIG. 2A, TIR 202a to TIR 202n, is associated with a different portion (i.e., sub-period of time, also referred to herein as a “block of time” or just a “block”) of period-of-interest 216 and arranged sequentially according to its associated portion. Graphical representation 228 has (e.g., visually depicts, without limitation) declinations (e.g., tick marks or white space arranged adjacent to tick marks, without limitation) that define boundaries of period-of-interest 216 and respective boundaries of blocks of time. In the specific example depicted by FIG. 2A, TIRs and associated portions of period-of-interest 216 are defined by Table 2:

| Time-In-Range (TIR) | Portion of Period-of-interest 216 |
|---|---|
| TIR 202a | Feb. 13 to Feb. 20 |
| TIR 202b | Feb. 20 to Feb. 27 |
| TIR 202c | Feb. 27 to Mar. 5 |
| TIR 202d | Mar. 5 to Mar. 12 |
| TIR 202e | Mar. 12 to Mar. 19 |
| TIR 202f | Mar. 19 to Mar. 26 |
| TIR 202g | Mar. 26 to Apr. 2 |
| TIR 202h | Apr. 2 to Apr. 9 |
| TIR 202i | Apr. 9 to Apr. 16 |
| TIR 202j | Apr. 16 to Apr. 23 |
| TIR 202k | Apr. 23 to Apr. 30 |
| TIR 202l | Apr. 30 to May 7 |
| TIR 202m | May 7 to May 14 |

Solely for convenience, in one or more examples, period-of-interest 216 should be understood to be divided into blocks of time that substantially correspond to diurnal rhythms' associated with insulin therapy for treating diabetes. Lengths of blocks of period-of-interest 216 may be chosen to provide (e.g., to a user) any desired resolution of change in quality of glucose control over time without exceeding the scope of this disclosure.

Notably, the arrangement of TIRs presented on graphical representation 228 facilitates comparison of the quality of glucose control represented by TIR 202a to TIR 202n, and by extension, for various blocks of period-of-interest 216. The outlines of respective TIRs of graphical representation 228 define respective areas having substantially a same measurement and shape. Further, TIRs are presented at graphical representation 228 in an “even row” by locating a bottom portion of their respective outlines along a common line that is substantially parallel to the x-axis of graphical representation 228. The substantially same positioning of respective TIRs relative to the x-axis of graphical representation 228 and the substantially same area measurements of the areas defined by respective outlines of the TIRs together provide a common reference for a user to visually compare the TIRs presented at graphical representation 228 over period-of-interest 216.

Changes in relative contribution to quality of glucose control by specific quality factors are depicted by graphical representation 228 via constituent bar charts of TIRs over period-of-interest 216. For example, a larger relative contribution by in-range bar chart 204c to a quality of glucose control represented by TIR 202c is depicted than a relative contribution by in-range bar chart 204d to quality of glucose control represented by TIR 202d. As such, TIR 202c and TIR 202d, together depict a change (here a decrease) in relative contribution of “time in-range” quality factor to a quality of glucose control. Moreover, a larger relative contribution of quality factors represented by level 2 hyper bar chart 208d, level 1 hyper bar chart 206d and level 2 hypo bar chart 212d to quality of glucose control is depicted than a relative contribution of quality factors represented by level 2 hyper bar chart 208c, level 1 hyper bar chart 206c, and level 2 hypo bar chart 212c to quality of glucose control. A user can quickly discern that more time was spent in level 2 hyperglycemia and in level 1 hypoglycemia during a period defined between March 5 and March 12 than in a period of time defined by March 12 and March 26.

In one or more examples, a number of indicators may be presented at graphical representation 228 to indicate a change in a therapy setting, such as a therapy setting related to long-acting insulin dosing or a therapy setting related to rapid-acting insulin dosing. Such indicators may be presented at a region of graphical representation 228 defined between the TIRs and x-axis 218, and at a location along x-axis 218 that corresponds to a time associated with the therapy setting change (e.g., a time a new setting was input to a dosing system, or a time at which a new setting first affected dosing, without limitation). For example, setting update indicator 220 is presented at graphical representation 228 and a time associated with setting update indicator 220 that is visually defined by looking down from setting update indicator 220 to a location on x-axis 218 substantially directly below setting update indicator 220. Such location on x-axis 218 corresponds to a time associated with a therapy setting change indicated by setting update indicator 220.

In one or more examples, an arrangement of therapy setting change indicators and TIRs presented at a graphical representation 228 may provide a user a visual representation of an effect of a therapy setting change on quality of glucose control, and more specifically, visually associate a therapy setting change with a change in quality of glucose control. For example, a relative increase in contribution of quality factors represented by in-range bar chart 204c and in-range bar chart 204e to quality of glucose control associated is visually associated with setting update indicator 220 by graphical representation 228.

In one or more examples, graphical user interface 214 may be configured to initiate presentation of information at graphical representation 228, for example, in response to an initiation event such as a pointer position or click (e.g., via a computer mouse or touch pad) or a touch location or tap (e.g., via a touch display) at a GUI element of graphical user interface 214. Such GUI elements may be linked to specific elements of graphical representation 228. For example, a GUI element may be generated and linked to setting update indicator 222 upon presentation of setting update indicator 222 at graphical representation 228, a GUI element may be generated and linked to TIR 202d upon presentation of TIR 202d, and a number of GUI elements may be generated and linked to constituent bar charts of TIR 202d upon their presentation.

FIG. 2B is a diagram depicting a specific example of display portion 200 that includes an information pane 230 presented over (i.e., visually in front of) graphical representation 228. Presentation of information pane 230 is initiated by graphical user interface 214 in response to an interaction event with an icon of setting update indicator 222, for example, hovering a pointer over an icon of setting update indicator 222.

Information pane 230 includes information relevant to relating a therapy setting change to changes in quality of glucose control represented by graphical representation 228 in accordance with one or more examples. In the specific example depicted by FIG. 2B, information pane 230 is configured to present information about a Meal Insulin Update in a column-row arrangement. More specifically, information pane 230 presents an update type heading of “Meal Insulin update”; presents column headings of “Meal Type,” “Previous,” “Change,” and “Units”; presents row headings, beneath the “Meal Type” heading of “Breakfast,” “Lunch,” and “Dinner,” and presents numbers corresponding to dosed units of rapid-acting insulin recorded for a previous day, dosed units of rapid-acting insulin recorded for a next day, and a change. An outcome of a therapy setting change was a change (an increase at breakfast and a decrease at dinner) in dosed units of rapid-acting insulin, and a possible effect of the change in dosed units of rapid-acting insulin was an increase in the relative contribution of quality factors represented by in-range bar chart 204d to quality of glucose control of the patient.

FIG. 2C is a diagram depicting a specific example of display portion 200 that includes an information pane 232 presented over (i.e., visually in front of) graphical representation 228 in accordance with one or more examples. Presentation of information pane 230 is initiated by graphical user interface 214 in response to an interaction event with a GUI element (such GUI element not labeled in FIG. 2C) defined with in-range bar chart 204a. In the specific example depicted by FIG. 2C, information pane 230 presents information about an average time in-range during a block (i.e., block of time) of period-of-interest 216 that is associated TIR 202a. More specifically, information pane 230 present an average time in-range, here “Avg: 10 h 17 min,” presents a percentage value of a total time of the block associated with TIR 202a, here “50%”; and presents a range of blood glucose values associated with time in-range, here, “70-180 mg/dL.”

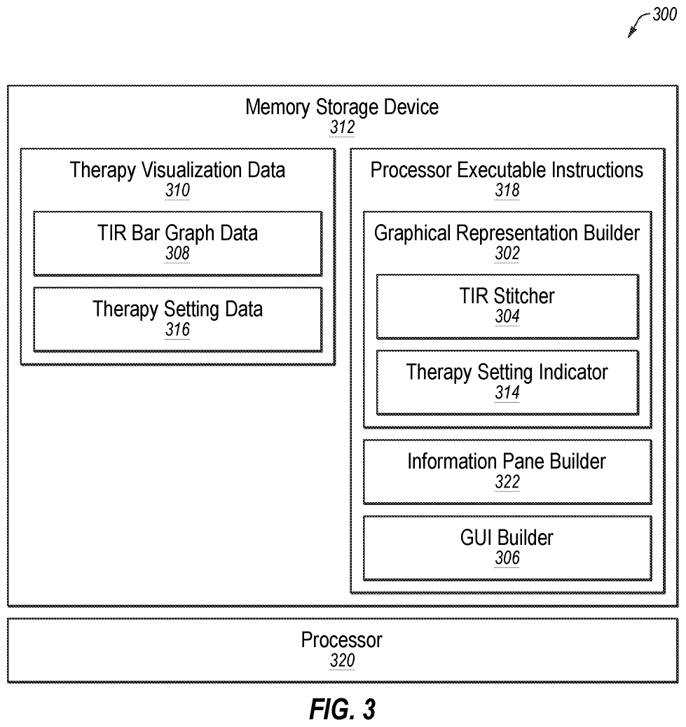

FIG. 3 is a block diagram depicting a computing system 300 configured to generate graphical representations and graphical user interfaces for visualizing changes in quality of glucose control (e.g., at a display, without limitation), in accordance with one or more examples. Computing system 300 includes memory storage device 312 and processor 320. Memory storage device 312 includes therapy visualization data 310 and processor executable instructions 318, which are adapted to enable executing processor 320 to perform some or a totality of the functions discussed herein with reference to various examples.

Therapy visualization data 310 includes, generally, data related to visualizing quality of glucose control and changes in quality of glucose control of a patient. Therapy visualization data 310 may include data for a specific period(s) of interest, or available data for a duration of a patient's insulin therapy from which data for a period-of-interest may be obtained as needed.

As depicted by FIG. 3, therapy visualization data 310 may include TIR bar graph data 308 and therapy setting data 316. TIRs of TIR bar graph data 308 may be defined for pre-specified blocks of time, and respective TIRs defined by the TIR bar graph data 308 may be stitched together for a given period-of-interest. TIRs defined by TIR bar graph data 308 individually represent quality of glucose control for a given block of time as well as depict the relative contribution of quality factors (see Table 1) to such quality of glucose control for a given block of time. Therapy setting data 316 includes information about therapy setting updates. In one or more examples, the information in therapy setting data 316 may be arranged according to changes in insulin dosing (e.g., with respect to a previous block of time) associated with changes to a therapy setting, such as the changes in amount of insulin dosed for Breakfast and Dinner presented at information pane 230 of FIG. 2B.

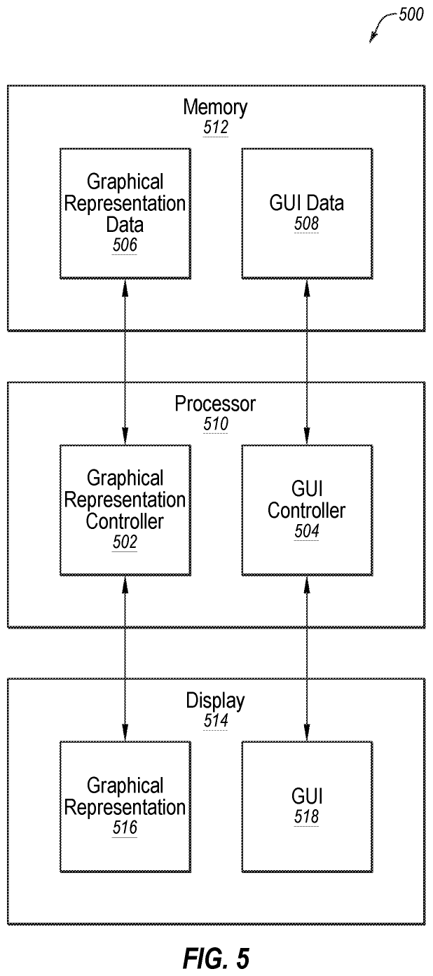

Processor executable instructions 318 include instructions, executable by processor 320, for generating a graphical representation and a GUI, such as a graphical representation 228 and a graphical user interface 214, without limitation. Processor executable instructions 318 include instructions for executing graphical representation builder 302 and instructions for executing GUI builder 306. Graphical representation builder 302 may be configured to generate graphical representation data (e.g., graphical representation data 506 of FIG. 5) of graphical representations. Graphical representation builder 302 may include TIR stitcher 304 and therapy setting indicator 314. TIR stitcher 304 may be configured to generate a layout for a graphical representation of the combined data of the respective TIRs to present respective TIRs together as discussed herein. Therapy setting indicator 314 may be configured to generate information for icons of update indicators and locations of update indicators (e.g., setting update indicator 220, setting update indicator 222, setting update indicator 224, and setting update indicator 226, without limitation).

Information pane builder 322 may be configured to assemble information for information panes that may be generated by a GUI when a user interacts with a TIR (e.g., information in information pane 232, without limitation), a setting update indicator (e.g., information in information pane 230, without limitation), or any other interaction that may be defined.

GUI builder 306 may be configured, generally, to generate GUI elements that overlay a graphical representation and facilitate interaction with the graphical representation. For example, GUI builder 306 may be configured to generate GUI element definitions and layouts of such GUI elements so that, when executing the GUI, a user of display portion 200 may interact with a TIR or setting update indicators to cause display of information panes such as information pane 230 and information pane 232.

Notably, therapy visualization data 310, including TIR bar graph data 308 and therapy setting data 316, may be stored at a same or different physical computer-readable memory as processor executable instructions 318, and a at a same or different computing apparatus as processor 320. In one or more examples, therapy visualization data 310, and more specifically, TIR bar graph data 308 and therapy setting data 316, may be received and stored at memory storage device 312 in response to a query (as a non-limiting example, executed by processor 320 and sent by communication equipment (not shown)) to a remote service configured to provide such data.

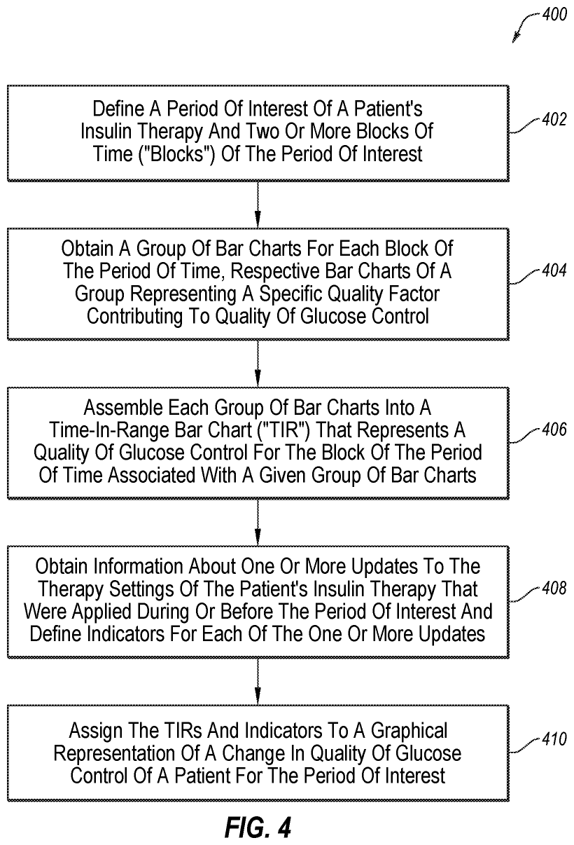

FIG. 4 is a flowchart depicting a process 400 for generating a graphical representation and GUI for visualizing changes in quality of glucose control in accordance with one or more examples.

At operation 402, process 400 defines a period-of-interest of a patient's insulin therapy and two or more blocks of time of the period-of-interest. As discussed, one or more available resolutions may be pre-specified, and/or a resolution may be chosen such that there is an option to define a resolution of the TIR for a given period-of-interest. If resolution is pre-specified then TIR bar graph data 308 stored in memory storage device 312 may be defined for specific blocks corresponding to a pre-specified resolution when process 400 begins. If a user chooses the resolution then therapy data, and more specifically glucose values, are processed to acquire time-in-range data for specific quality factors according to the desired resolution and then the processed data is stored at memory storage device 312 as TIR bar graph data 308.

At operation 404, process 400 obtains a group of bar charts for each block of the period of time. The respective bar charts of a group represent specific quality factors that contribute to quality of glucose control.

At operation 406, process 400 assembles each group of bar charts into a time-in-range bar chart (TIR) that represents a quality of glucose control for the block of the period of time associated with a given group of bar charts.

At operation 408, process 400 obtains information about one or more updates to the therapy settings and defines indicators for each of the one or more updates. The one or more updates may be insulin therapy updates for the patient's insulin therapy that were applied during or before the period-of-interest. Information about the one or more updates may include information about one or more of when the setting update occurred, when the setting update was first applied, or when a dosing event was first affected by the setting update, without limitation.

At operation 410, process 400 assigns the TIRs and indicators to a graphical representation of a change in quality of glucose control of a patient for the period-of-interest.

FIG. 5 is a block diagram depicting a computing apparatus 500 for presenting a graphical representation of changes in quality of glucose control, in accordance with one or more examples. Computing apparatus 500 includes processor 510, memory 512, and display 514 and more specifically, computing apparatus 500 comprises a graphical representation controller 502, a GUI controller 504 (e.g., a “further controller,” without limitation), a graphical representation data 506, a GUI data 508, a processor 510, a memory 512, a display 514, a graphical representation 516, and a GUI 518.

Graphical representation controller 502 may be configured, generally, to control presentation of graphical representation 516 at display 514 in response to graphical representation data 506 stored at memory 512. Graphical representation data 506 may be a data for a graphical representation generated by computing system 300 and in response to process 400.

GUI controller 504 may be configured, generally, to control presentation of GUI 518 at display 514 in response to GUI data 508 stored at memory 512 and thereby facilitate user interaction with graphical representation 516. GUI data 508 may include the GUI element definitions and layout generated by GUI builder 306 and the information panes generated by information pane builder 322.

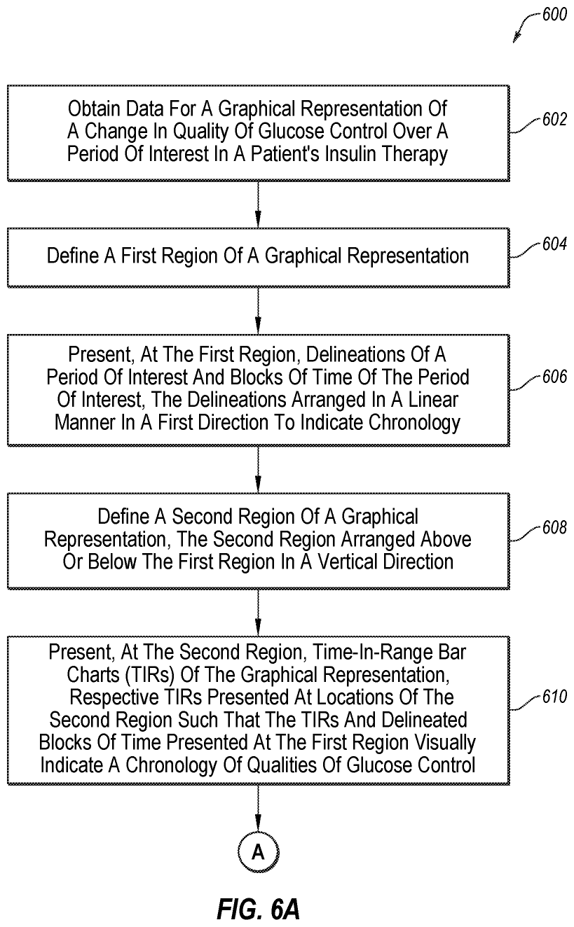

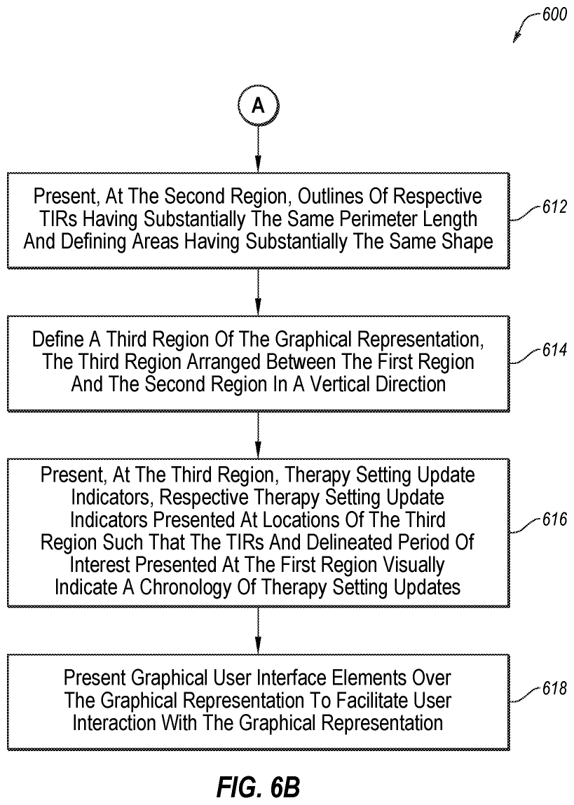

FIGS. 6A and 6B are a flowchart depicting a process 600 for visualizing changes in quality of glucose control, in accordance with one or more examples.

At operation 602, process 600 obtains data for a graphical representation of a change in quality of glucose control of a period-of-interest in a patient's insulin therapy.

At operation 604, process 600 defines a first region of the graphical representation. The first region may be a 2-dimensional area corresponding to a portion of a display.

At operation 606, process 600 presents, at the first region of the graphical representation, delineations of a period-of-interest and blocks of time of the period-of-interest. The delineations may be arranged in a linear manner in a first direction to indicate a chronology.

At operation 608, process 600 defines a second region of the graphical representation. The second region may also be a 2-dimensional area corresponding to a portion of a display. The second region may be arranged above or below the first region in a vertical direction as would be viewed by a user.

At operation 610, process 600 presents, at the second region of the graphical representation, time-in-range bar charts (TIRs) of the graphical representation. Respective TIRs are presented at locations of the second region such that the TIRs and delineated blocks of time presented at the first region visually indicate a chronology of qualities of glucose control for a patient. For example, FIG. 2A depicts each TIR 202a-TIR 202n above a space defined by two vertical lines, and each vertical line has a date presented to its right. The width of each TIR (in a horizontal direction as would be viewed by a user) is confined substantially to a width of the space defined by the two vertical lines. Further, in the specific example depicted by FIG. 2A, spaces defined between respective TIRs visually align in the vertical direction with the aforementioned vertical lines.

At operation 612, process 600 presents, at the second region, outlines of respective TIRs having substantially a same perimeter length and defining areas having substantially the same dimensions. For example, FIG. 2A depicts each outline of a TIR having a substantially rectangular shape having the same dimensions (e.g., visually have substantially the same length in the vertical direction and same width in a horizontal direction, without limitation). Moreover, as discussed above, TIRs are presented by process 600, in an “even row” such that the upper or lower boarders of depicted contributions for a given quality factor visually form discontinuous elements of a continuous curve.

At operation 614, process 600 defines a third region of the graphical representation. The third region may also be a 2-dimensional area corresponding to a portion of a display. The third region may be arranged between the first region and the second region in a vertical direction with respect to the first region and the second region.

At operation 616, process 600 presents, at the third region, therapy setting update indicators. Respective therapy setting update indicators are presented at locations of the third region such that the TIRs and delineated period-of-interest presented at the first region visually indicate a chronology of therapy setting updates relative to the TIRs. For example, FIG. 2A depicts a setting update indicator 220 presented below TIR 202d and before (as indicated by the indicators location relative to the chronological information of x-axis 218) TIR 202e. The chronology of quality of glucose control represented by the TIRs that follow (in time) setting update indicator 220 visually indicate outcomes of therapy setting updates corresponding to setting update indicator 220, and more specifically, changes to specific quality factors that contribute to quality of glucose control.

At operation 618, process 600 presents graphical user interface elements of a graphical user interface over the graphical representation to facilitate user interaction with the graphical representation and further assist with visualizing changes in quality of glucose control and outcomes of applying therapy setting updates.

While time-in-range has been discussed herein with respect to one or more examples, a person having ordinary skill in the art would understand that this disclosure is not limited to just time-in-range type data. As a non-limiting example, time-out-of-range data and time-out-of-range bar carts may be used to depict changes in quality of a patient's glucose control, without exceeding the scope of this disclosure. Indeed, a person having ordinary skill in the art would appreciate that a TIR could be characterized as a time-out-of-range bar chart since it presents time-out-of-range information.

As used herein, the singular forms following “a,” “an,” and “the” are intended to include the plural forms as well, unless the context clearly indicates otherwise.

As used herein, the term “may” with respect to a material, structure, feature, function, or method act indicates that such is contemplated for use in implementation of an example of the disclosure, and such term is used in preference to the more restrictive term “is” so as to avoid any implication that other compatible materials, structures, features, functions, and methods usable in combination therewith should or must be excluded. As used herein “each” means some or a totality. As used herein, “each and every” means a totality.

As used herein, any relational term, such as “first,” “second,” etc., is used for clarity and convenience in understanding the disclosure and accompanying drawings, and does not connote or depend on any specific preference or order, except where the context clearly indicates otherwise.

As used herein, the term “substantially” in reference to a given parameter, property, act, or condition means and includes to a degree that one skilled in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measure of the given parameter, as well as variations resulting from manufacturing tolerances, etc.).

Additional non-limiting examples of the disclosure include:

Example 1: A computing apparatus for operative coupling to a display or including a display, the computing apparatus comprising: a controller to control presentation of a graphical representation at the display, the graphical representation having: a delineation of a period-of-interest presented at a first region of the graphical representation; and a representation of change in quality of glucose control presented at a second region of the graphical representation.

Example 2: The computing apparatus according to Example 1, wherein the representation of change in quality of glucose control having: two or more representations of quality of glucose control, each of the two or more representations of quality of glucose control associated with a given block of time of the period-of-interest.

Example 3: The computing apparatus according to Examples 1 and 2, wherein the representation of change in quality of glucose control comprising: a first representation of quality of glucose control and a second representation of quality of glucose control presented at the second region and arranged chronologically.

Example 4: The computing apparatus according to Examples 1 to 3, wherein the first and second representation of quality of glucose control each comprising portions that individually visually represent a contribution of a specific quality factor to the quality of glucose control represented by respective ones of the first and second representation of quality of glucose control.

Example 5: The computing apparatus according to Examples 1 to 4, wherein respective outlines of the first and second representations of quality of glucose control each define areas having substantially same dimensions.

Example 6: The computing apparatus according to Examples 1 to 5, wherein the first and second representations of quality of glucose control are arranged in an even row.

Example 7: The computing apparatus according to Examples 1 to 6, wherein the graphical representation further having: one or more indicators presented at a third region of the graphical representation, the indicators configured to visually indicate a therapy setting update.

Example 8: The computing apparatus according to Examples 1 to 7, further comprising: a further controller configured to control presentation of a graphical user interface at the display, the graphical user interface having one or more graphical user interface elements for interacting with the graphical representation.

Example 9: The computing apparatus according to Examples 1 to 8, wherein a graphical user interface element is configured to initiate presentation of an information pane including information about a therapy setting update.

Example 10: The computing apparatus according to Examples 1 to 9, wherein a graphical user interface element is configured to initiate presentation of an information pane including information about a quality factor contributing to a quality of glucose control for a given block of time of the period-of-interest.

Example 11: A computing apparatus, comprising: a display; a controller configured to control presentation of a first graphical representation at the display, the first graphical representation having: a delineation of a period-of-interest presented at a first region of the graphical representation; and a second graphical representation, presented at a second region of the first graphical representation, the second graphical representation of an effectiveness of an insulin therapy setting update for changing a quality of glucose control of a patient.

Example 12: A method of generating a graphical representation of a change in quality of glucose control of a patient, comprising: defining a period of interest of a patient's insulin therapy and two or more blocks of time of the period of interest; obtaining groups of bar charts by obtaining a group of bar charts for each block of the period of time, respective bar charts of the group representing a specific quality factor contributing to quality of glucose control; assembling groups of bar charts into a time-in-range bar chart (“TIR”) that represents a quality of glucose control for the block of the period of time associated with a given group of bar charts; obtaining information about one or more updates to the therapy settings of the patient's insulin therapy that were applied during or before the period of interest and define indicators for each of the one or more updates; and assigning the TIRs and indicators to a graphical representation of a change in quality of glucose control of a patient for the period of interest.

Example 13: A method, comprising: obtaining data for a graphical representation of a change in quality of glucose control over a period of interest in a patients insulin therapy; defining a first region of a graphical representation; presenting, at the first region, delineations of a period of interest and blocks of time of the period of interest, the delineations arranged in a linear manner in a first direction to indicate chronology; defining a second region of a graphical representation, the second region arranged above or below the first region in a vertical direction; presenting, at the second region, time-in-range bar charts (TIRs) of the graphical representation, respective TIRs presented at locations of the second region such that the TIRs and delineated blocks of time presented at the first region visually indicate a chronology of qualities of glucose control; defining a third region of the graphical representation, the third region arranged between the first region and the second region in a vertical direction; and presenting, at the third region, therapy setting update indicators, respective therapy setting update indicators presented at locations of the third region such that the TIRs and delineated period of interest presented at the first region visually indicate a chronology of therapy setting updates.

Example 14: The method according to Example 13, further comprising: presenting a graphical user interface element over the graphical representation.

Example 15: The method according to Examples 13 and 14, further comprising: facilitating user interaction with the graphical representation via the graphical user interface.

The examples of the disclosure described above and illustrated in the accompanying drawing figures do not limit the scope of the invention, since these examples are merely examples of examples of the invention, which is defined by the appended claims and their legal equivalents. Any equivalent examples are intended to be within the scope of this invention. Indeed, various modifications of the present disclosure, in addition to those shown and described herein, such as alternative useful combinations of the content features described, may become apparent to those skilled in the art from the description. Such modifications and examples are also intended to fall within the scope of the appended claims and legal equivalents.

What is claimed is:

1. A computing apparatus for operative coupling to a display or including a display, the computing apparatus comprising:
a controller to control presentation of a graphical representation at the display, the graphical representation having:
a delineation of a period-of-interest presented at a first region of the graphical representation; and
a representation of change in quality of glucose control presented at a second region of the graphical representation.

2. The computing apparatus of claim 1, wherein the representation of change in quality of glucose control having: two or more representations of quality of glucose control, each of the two or more representations of quality of glucose control associated with a given block of time of the period-of-interest.

3. The computing apparatus of claim 1, wherein the representation of change in quality of glucose control comprising: a first representation of quality of glucose control and a second representation of quality of glucose control presented at the second region and arranged chronologically.

4. The computing apparatus of claim 3, wherein the first and second representation of quality of glucose control each comprising portions that individually visually represent a contribution of a specific quality factor to the quality of glucose control represented by respective ones of the first and second representation of quality of glucose control.

5. The computing apparatus of claim 3, wherein respective outlines of the first and second representations of quality of glucose control each define areas having substantially same dimensions.

6. The computing apparatus of claim 3, wherein the first and second representations of quality of glucose control are arranged in an even row.

7. The computing apparatus of claim 1, wherein the graphical representation further having:
one or more indicators presented at a third region of the graphical representation, the indicators configured to visually indicate a therapy setting update.

8. The computing apparatus of claim 1, further comprising:
a further controller configured to control presentation of a graphical user interface at the display, the graphical user interface having one or more graphical user interface elements for interacting with the graphical representation.

9. The computing apparatus of claim 8, wherein a graphical user interface element is configured to initiate presentation of an information pane including information about a therapy setting update.

10. The computing apparatus of claim 8, wherein a graphical user interface element is configured to initiate presentation of an information pane including information about a quality factor contributing to a quality of glucose control for a given block of time of the period-of-interest.

11. A computing apparatus, comprising:
a display;
a controller configured to control presentation of a first graphical representation at the display, the first graphical representation having:
a delineation of a period-of-interest presented at a first region of the graphical representation; and
a second graphical representation, presented at a second region of the first graphical representation, the second graphical representation of an effectiveness of an insulin therapy setting update for changing a quality of glucose control of a patient.

12. A method of generating a graphical representation of a change in quality of glucose control of a patient, comprising:
defining a period of interest of a patient's insulin therapy and two or more blocks of time of the period of interest;
obtaining groups of bar charts by obtaining a group of bar charts for each block of the period of time, respective bar charts of the group representing a specific quality factor contributing to quality of glucose control;
assembling groups of bar charts into a time-in-range bar chart (“TIR”) that represents a quality of glucose control for the block of the period of time associated with a given group of bar charts;
obtaining information about one or more updates to therapy settings of the patient's insulin therapy that were applied during or before the period of interest and define indicators for each of the one or more updates; and
assigning the TIRs and indicators to a graphical representation of a change in quality of glucose control of a patient for the period of interest.

13. A method, comprising:
obtaining data for a graphical representation of a change in quality of glucose control over a period of interest in a patients insulin therapy;
defining a first region of a graphical representation;
presenting, at the first region, delineations of a period of interest and blocks of time of the period of interest, the delineations arranged in a linear manner in a first direction to indicate chronology;
defining a second region of a graphical representation, the second region arranged above or below the first region in a vertical direction;
presenting, at the second region, time-in-range bar charts (TIRs) of the graphical representation, respective TIRs presented at locations of the second region such that the TIRs and delineated blocks of time presented at the first region visually indicate a chronology of qualities of glucose control;
defining a third region of the graphical representation, the third region arranged between the first region and the second region in a vertical direction; and
presenting, at the third region, therapy setting update indicators, respective therapy setting update indicators presented at locations of the third region such that the TIRs and delineated period of interest presented at the first region visually indicate a chronology of therapy setting updates.

14. The method of claim 13, further comprising:
presenting a graphical user interface elements over the graphical representation.

15. The method of claim 14, further comprising:
facilitating user interaction with the graphical representation via the graphical user interface.
